(19) United States
(12) Patent Application Publication
(10) Pub. No.: US 2006/0078624 A1
(43) Pub. Date: Apr. 13, 2006

(54) Microparticles and nanoparticles containing a lipopolymer

(76) Inventors: Samuel Zalipsky, Redwood City, CA (US); Joyce Yun-Wei Wong, Chestnut Hill, MA (US)

Correspondence Address: PERKINS COIE LLP, P.O. BOX 2168, MENLO PARK, CA, 94026, US

(21) Appl. No.: 11/238,542

(22) Filed: Sep. 28, 2005

Related U.S. Application Data
(60) Provisional application No. 60/614,537, filed on Sep. 29, 2004.

(51) Int. Cl. A61K 9/14; A61K 38/17

(52) U.S. Cl. 424/489; 514/2

(57) ABSTRACT

Microparticle and nanoparticles comprised of a polymer and of a lipid-polymer conjugate (lipopolymer) are described. The particles can include a therapeutic or diagnostic agent and/or a ligand attached to the lipopolymer. Compositions comprising the particles and methods of treatment using the particles are also described.

### CROSS-REFERENCE TO RELATED APPLICATIONS

This application claims the benefit of U.S. Provisional Application No. 60/614,537, filed Sep. 29, 2004, incorporated herein by reference in its entirety.

### TECHNICAL FIELD

The subject matter described herein relates to particles, particularly nanoparticles and microparticles, comprised of a biodegradable polymer and of a lipopolymer. The particles can include a ligand attached to all or a fraction of the lipopolymer conjugates, where the ligand can, for example, target the particles to a specific tissue in vivo or serve as a diagnostic agent. More generally, the subject matter described herein relates to a composition comprised of such particles for delivering an agent, such as a therapeutic agent or a diagnostic agent, to a cell.

### BACKGROUND

The use of nanoparticles and microparticles has found application in a variety of disciplines, including the use of such particles in pharmacology and drug delivery. Nanoparticles as carriers of anticancer and other drugs was proposed long ago (Couvreur et al., J. Pharm. Sci., 71:790-92 (1982)) followed by attempts to elucidate methods by which the uptake of the nanoparticles by the cells of the reticuloendothelial system (RES) would be minimized (Couvreur et al., in POLYMERIC NANOPARTICLES AND MICROSPHERES, (Guiot & Couvreur, eds.), CRC Press, Boca Raton, pp. 27-93 (1986); Illum, L. et al., FEBS Lett., 167(1):79 (1984)).

Although nanoparticles and microparticles have shown promise as useful tools for drug delivery systems, many problems remain. Some unsolved problems relate to the loading of therapeutic agents into the particles, the rate of release of the agent, and the circulation lifetime of the particles. Additionally, the targeting of the particles to a desired in vivo site has remained problematic. The development of new forms of therapeutics that use macromolecules such as proteins or nucleic acids as therapeutic agents has created a need to develop new and effective approaches of delivering such macromolecules to their appropriate cellular targets. The development of improved chemotherapeutic agents has increased the need for site specific delivery of the agent. Clinical use of these new therapeutics depends not only on the reliability and efficiency of the delivery systems but also on the safety and on the ease with which the delivery system can be adapted for large-scale pharmaceutical production and storage.

Particles bearing a ligand for targeting have been proposed (see U.S. Pat. Nos. 5,543,158; 5,565,215; 5,578,325; 6,007,845; US 2003/0223938). The prior art approaches typically involve preparation of particles from a hydrophilic-hydrophobic block copolymer, where the hydrophilic block can be conjugated to a targeting ligand (U.S. Pat. Nos. 5,543,158; 5,565,215; 5,578,325; 6,007,845). For example, micelles and particles formed from block copolymers of poly(lactic acid) and polyethylene glycol, where the polyethylene glycol bears a terminal ligand, have been described (Yasugi, K. et al., Macromolecules, 32:8024 (1999); Oliver, J-C. et al., Pharm. Res., 19:1137 (2002)). Problems remain with this approach, however. Preparation of block copolymers typically results in a polydisperse material, with best M_{w}/Mₙ ratios in 1.2 range, but often higher. Moreover, such block copolymers are not readily available commercially with additional reactive functionalities that are needed for attachment of ligands to the free polymer end. Thus, custom synthesis is often required which is costly. When a biodegradable polymer such as poly(lactic acid) is used as one of the copolymer blocks, there are problems associated with stability of the particle. Poly(lactic acid) is a bioerodible polyester. When a particle is formed from a poly(lactic-acid)-based copolymer, e.g., poly(lactic-acid)-polyethylene glycol (PEG), the PEG portion of the block copolymer is exposed to the external aqueous environment. The ester linkage between the PEG block and the poly(lactic acid) is vulnerable to hydrolysis due to its proximity with the aqueous surroundings. This can result in a premature detachment of the polyethylene glycol from the particle, and loss of extended circulation time offered by the polyethylene glycol chains and/or loss of a ligand linked to the PEG block.

The foregoing examples of the related art and limitations related therewith are intended to be illustrative and not exclusive. Other limitations of the related art will become apparent to those of skill in the art upon a reading of the specification and a study of the drawings.

### SUMMARY

Accordingly, microparticles and nanoparticles capable of serving as a carrier vehicle for delivery of an agent to a specific site in vivo are described.

In one aspect, a nanoparticle or a microparticle comprised of a biodegradable polymer and of a lipid-hydrophilic polymer-ligand (“lipopolymer-ligand”) conjugate, where the lipopolymer-ligand conjugate is stably incorporated into the nanoparticle or microparticle to provide an outer surface coating of hydrophilic polymer chains and a ligand accessible for interaction with a binding partner.

In one embodiment, the biodegradable polymer is selected from poly(lactic acid), poly(glycolic acid), poly(lactic-co-glycolic acid), and copolymers prepared from monomers of these polymers.

In another embodiment, the lipid-hydrophilic polymer conjugate is a lipid-poly(alkylene glycol) conjugate.

The nanoparticle or microparticle can optionally include an agent, such as a therapeutic agent or a diagnostic agent. The agent can be incorporated into the particle or associated with the particle. Exemplary agents include proteins, peptides, and organic compounds.

The nanoparticles or microparticles can include one or more of the same or different ligands, attached to all or a portion of hydrophilic polymer chains surrounding the particle. That is, a single particle can have two or more different ligands attached to the hydrophilic polymer chains. Exemplary ligands include biologically active ligands, targeting ligands, and diagnostic ligands.

Also described, in another aspect, is a method for delivering an agent to a subject, comprising administering to the subject a composition comprising nanoparticles or microparticles as described above.

In one embodiment, the nanoparticles or microparticles comprise an agent having therapeutic activity.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1B are brightfield and fluorescence images, respectively, of microparticles comprised of poly(lactic acid) and a fluorescently-labeled lipid (Scale bars are 100 μm);

FIGS. 1C-1D are brightfield and fluorescence images, respectively, of microparticles comprised of poly(lactic acid) (scale bars are 100 μm);

FIGS. 2A-2C are brightfield images of microparticles comprised of poly(lactic acid) and a fluorescently-labeled lipid after washing with water (FIG. 2A), after washing once with sodium dodecyl sulfate (FIG. 2B), and after washing three times with sodium dodecyl sulfate (FIG. 2C) (scale bars are 100 μm);

FIGS. 2D-2F are fluorescence images of microparticles comprised of poly(lactic acid) and a fluorescently-labeled lipid after washing with water (FIG. 2D), after washing once with sodium dodecyl sulfate (FIG. 2E), and after washing three times with sodium dodecyl sulfate (FIG. 2F) (scale bars are 100 μm);

FIGS. 3A-3C are brightfield images of poly(lactic acid) microparticles with lipid physisorbed onto the preformed microparticles, after washing with water (FIG. 3A), after washing once with sodium dodecyl sulfate (FIG. 3B), and after washing three times with sodium dodecyl sulfate (FIG. 3C) (scale bars are 100 μm);

FIGS. 3D-3F are fluorescence images of poly(lactic acid) microparticles with lipid physisorbed onto the preformed microparticles, after washing with water (FIG. 3D), after washing once with sodium dodecyl sulfate (FIG. 3E), and after washing three times with sodium dodecyl sulfate (FIG. 3F) (scale bars are 100 μm);

FIGS. 4A-4C are brightfield images of poly(lactic acid) microparticles incubated with fluorescently-labeled streptavidin and with bovine serum albumin, where the microparticles are comprised of poly(lactic acid) alone (FIG. 4A), of poly(lactic acid) and mPEG-DSPE (FIG. 4B) or of poly(lactic acid) and biotin-PEG-DSPE (FIG. 4C) (Scale bars are 25 μm);

FIGS. 4D-4F are fluorescence images of poly(lactic acid) microparticles incubated with fluorescently-labeled streptavidin and with bovine serum albumin, where the microparticles are comprised of poly(lactic acid) alone (FIG. 4D), of poly(lactic acid) and mPEG-DSPE (FIG. 4E) or of poly(lactic acid) and biotin-PEG-DSPE (FIG. 4F) (scale bars are 25 μm); and

FIG. 5 is a bar graph of microparticle density per 100,000 micrometers of an eggPC-streptavidin or eggPC substrate for biotin-labeled microparticle compositions (dotted bars) and for thioctic acid-containing microparticle compositions (hatched bars), the compositions containing, in addition to the biotin- or thioctic acid-polymer (PEG₃₃₀₀)-lipid conjugate, a polymer-lipid conjugate, where the polymer was PEG with a molecular weight of 2000 or 5000 daltons.

### DETAILED DESCRIPTION

### I. DEFINITIONS

The term “nanoparticle” as used herein denotes a structure ranging in size from 1 to 1000 nanometer (nm), and preferably having any diameter less than or equal to 1000 nm, including 5, 10, 15, 20, 25, 30, 50, 100, 500 and 750 nm.

The term “microparticle” as used herein intends a structure ranging in size from about 1 micrometer (μm) to about 1000 μm; and preferably having any diameter less than or equal to 1000 μm, including 5, 10, 15, 20, 25, 30, 50, 100, 500 and 750 μm.

As used herein, the term “agent” means a therapeutic agent or a diagnostic agent, examples of which are given below, but generally encompass any agent used for purposes of preventing, treating, ameliorating, a disorder, a condition, a disease, and/or symptoms associated therewith, or detecting or diagnosing a disorder, condition, or disease. The agent can be incubated with the particles for adsorption or attachment to the particle, or admixed with the polymer during particle formation for incorporation into the core of the particle.

“Lipopolymer” is used interchangeably with “lipid-polymer” and “lipid-hydrophilic polymer” and intends a hydrophobic moiety covalently attached to a hydrophilic polymer chain. A “lipid-polymer-ligand” or “lipopolymer-ligand” refers to a lipopolymer having an attached or associated ligand. Typically, the ligand is attached to the distal free terminus of the polymer, but could be attached to a side chain or branch on the polymer. The ligand can be, for example, a biologically relevant moiety, a diagnostic compound, a reactive moiety, a therapeutic agent, etc.

A “hydrophilic polymer” intends a polymer having some amount of solubility in water at room temperature. Exemplary hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers. Preferred polymers are polyalkylene glycol, such as polyethyleneglycol (PEG), preferably as a PEG chain having a molecular weight between 500-10,000 daltons, more preferably between 750-10,000 daltons, still more preferably between 750-5000 daltons.

The term “therapeutic agent” intends any agent having a therapeutic effect.

As used herein, the term “biodegradable” means any structure that decomposes or otherwise disintegrates after prolonged exposure to physiological conditions. To be biodegradable, the structure should be substantially disintegrated within a few weeks after introduction into the body.

As used herein, the terms “cellular targeting ligand” or “extracellular targeting ligand” are used interchangeably and refer to a small molecule or protein sequence that is recognized and bound by one or more receptors present on the surface of a particular cell. It is preferable, but not required, that a “cellular targeting ligand” that is recognized and/or bound by a cell surface receptor leads to internalization via receptor-mediated endocytosis. Representative moieties that can be employed as targeting ligands for internalization are provided below.

The term “pharmaceutically acceptable” intends materials are capable of administration to a vertebrate subject without the production of undesirable physiological effects, such as nausea, dizziness, gastric upset, fever and the like.

As used herein, the terms “polypeptide”, “protein”, and “peptide” are used interchangeably and mean any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted.

As used herein, the term “small molecule” means a molecule that has a molecular weight of less than or equal to 5000 Daltons, more typically less than 1000 Daltons, and is generally used in the context of a small molecule drug (therapeutic agent) as distinguished from a polypeptide therapeutic agent.

### II. PARTICLES AND COMPOSITIONS COMPRISING PARTICLES

In one aspect, particles comprised of a biodegradable polymer and of a lipopolymer are provided. The term “particles” will be used generally to refer to a population of nanoparticles, to a population of microparticles, or to a population of nanoparticles and microparticles. Where needed, specific reference to ‘nanoparticles’ or ‘microparticles’ will be made. The particles are formed from a biodegradable polymer, and more preferably from a pharmaceutically-acceptable biodegradable polymer. The particles also include a lipopolymer, that is a lipid-hydrophilic polymer conjugate, and preferably, a lipopolymer-ligand conjugate. These various components and examples of particles will now be described.

The particles described herein can be prepared from non-biodegradable or biodegradable polymers, however, biodegradable polymers are preferred. The polymer may be natural or synthetic, with synthetic polymers being preferred due to the better characterization of degradation and, where appropriate, release profile of an incorporated agent. The polymer is selected based on the period over which degradation or release of an agent is desired, generally in the range of at several weeks to several months, although shorter or longer periods may be desirable.

Representative biodegradable polymers include synthetic polymers such as polymers of lactic acid and glycolic acid, polyanhydrides, poly(ortho)esters, polyurethanes, poly(hydroxybutiric acid), poly(valeric acid), and poly (lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin, and other hydrophilic proteins. The particles can also be formed from bioerodible hydrogels which are prepared from materials and combinations of materials such as polyhyaluronic acids, casein, gelatin, glutin, polyanhydrides, polyacrylic acid, alginate, chitosan, poly(methyl methacrylates), poly(ethyl methacrylates), poly(butylmethacrylate), poly (isobutyl methacrylate), poly (hexylmethacrylate), poly (isodecyl methacrylate), poly (lauryl methacrylate), poly (phenyl methacrylate), poly(methyl acrylate), poly(isopropyl acrylate), poly(isobutyl acrylate), and poly(octadecyl acrylate). Preferred biodegradable polymers are polyglycolic acid, polylactic acid, copolymers of glycolic acid and L- or D,L-lactic acid, and copolymers of glycolide and L- or D,L-lactide. Those of skill in the art will appreciate that the molecular weight of the polymer can be varied to tailor the properties of the particle.

The foregoing exemplary natural and synthetic polymers are, of course, either readily available commercially or are obtainable by condensation polymerization reactions from the suitable monomers, comonomers, or oligomers. For instance, homopolymers and copolymers of glycolic and lactic acids can be prepared by direct poly-condensation or by reacting glycolide and lactide monomers (Gilding, D. K., et al., Polymer, 20:1459 (1979)).

The particles described herein also include a conjugate of a lipid and a hydrophilic polymer, referred to as a ‘lipopolymer.’ Lipopolymers can be obtained commercially or can be synthesized using known procedures. For example, lipopolymers comprised of methoxy(polyethylene glycol) (mPEG) and a phosphatidylethanolamine (e.g., dimyristoyl phosphatidylethanolamine, dipalmitoyl phosphatidylethanolamine, 1,2-distearoyl-3-sn-glycerophosphoethanolamine (distearoyl phosphatidylethanolamine (DSPE)), or dioleoyl phosphatidylethanolamine) can be obtained from Avanti Polar Lipids, Inc. (Alabaster, Ala.) at various mPEG molecular weights (350, 550, 750, 1000, 2000, 3000, 5000 Daltons). Lipopolymers of mPEG-ceramide can also be purchased from Avanti Polar Lipids, Inc. Preparation of lipid-polymer conjugates is also described in the literature, see U.S. Pat. Nos. 5,631,018, 6,586,001, and 5,013,556; Zalipsky, S. et al., Bioconjugate Chem., 8:111 (1997); Zalipsky, S. et al., Meth. Enzymol., 387:50 (2004). These lipopolymers can be prepared as well-defined, homogeneous materials of high purity, with minimal molecular weight dispersity (Zalipsky, S. et al., Bioconjugate Chem., 8:111 (1997); Wong, J. et al., Science, 275:820 (1997)). The lipopolymer can also be a “neutral” lipopolymer, such as a polymer-distearoyl conjugate, as described in U.S. Pat. No. 6,586,001, incorporated by reference herein.

The hydrophobic component of the lipopolymer can be virtually any hydrophobic compound having or modified to have a chemical group suitable for covalent attachment of a hydrophilic polymer chain. Exemplary chemical groups are, for example, an amine group, a hydroxyl group, an aldehyde group, and a carboxylic acid group. Preferred hydrophobic components are lipids, such as cholesterol, cholesterol derivatives, sphingomyelin, and phospholipids, such as phosphatidylcholine (PC), phosphatidylethanolamine (PE), phosphatidylglycerol (PG), phosphatidic acid (PA), phosphatidylinositol (PI), where the two hydrocarbon chains are typically between about 8-24 carbon atoms in length, and have varying degrees of unsaturation. These lipids are exemplary and not intended to be limiting, as those of skill can readily identify other lipids that can be covalently modified with a hydrophilic polymer and incorporated into the particles described herein. A preferred lipopolymer is formed of polyethylene-glycol and a lipid, such as distearoyl phosphatidylethanolamine (DSPE), PEG-DSPE. PEG-DSPE has some degree of biodegradability in vivo, by virtue of the hydrolysable bonds between the fatty acids and the glycerol moiety. When the PEG-lipid is incorporated into a particle, the hydrolysable bond is in a water-free environment and thus stabilized. The linkage between the PEG and the lipid can be stable or labile as desired. For example, a more stable urethane linkage can join the polymer to the lipid, and in this case the PEG-lipid will be stably incorporated in the particle until the particle is essentially eroded.

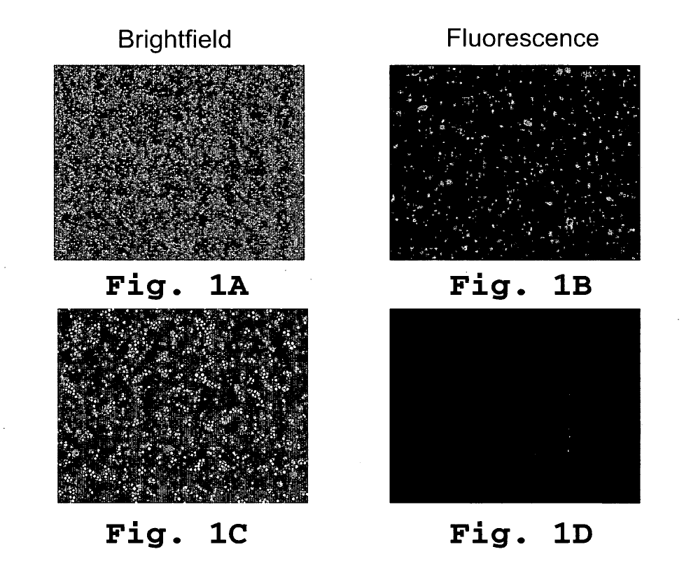

A study was performed to show that a lipid can be incorporated into particles prepared from an exemplary biodegradable polymer, poly(dl-lactide). As described in Example 1, microparticles and nanoparticles prepared from poly (lactic acid) and the lipid 1,2-dihexadecanoyl-sn-glycero-3-phosphoethanolamine (DHPE) labeled with Texas Red® were prepared by dissolving the polymer in a suitable organic solvent and adding the labeled lipid to the mixture. Addition of a surfactant and homogenization produced microparticles, which were recovered. Phase-contrast microscopy was used to verify the formation of the microspheres and fluorescence microscopy was used to assess lipid incorporation and protein binding. The photomicrographs are shown in FIGS. 1A-1D.

FIGS. 1A-1B show brightfield and fluorescence images, respectively, of the poly(lactic acid)-lipid microspheres. The fluorescent image verifies that Texas Red®-X-DHPE can be incorporated into the polymeric microparticles. For comparison to these microparticles having an incorporated lipid, microparticles of pure poly(lactic acid) were prepared according to the same procedure, and the brightfield and fluorescence images of these control particles are shown in FIGS. 1C-1D, respectively.

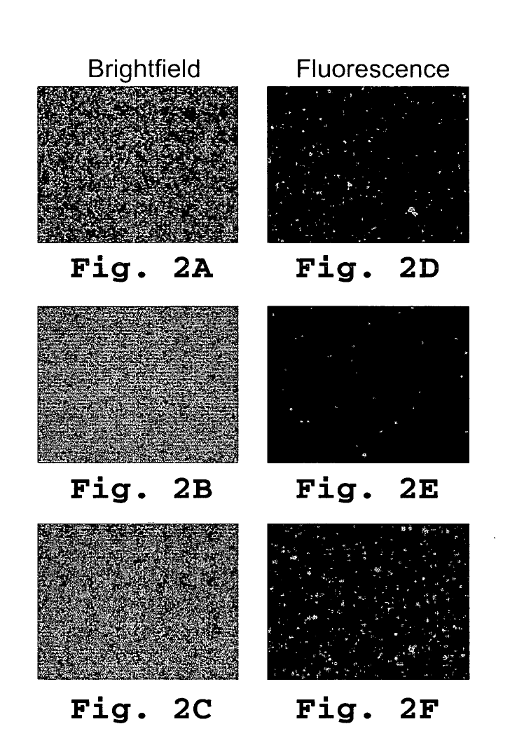

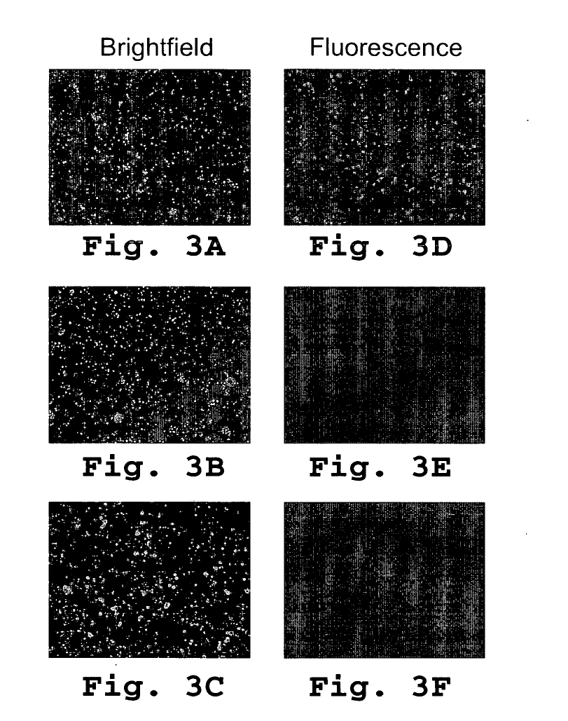

The stability of incorporation of the lipopolymer was evaluated by further analysis of the DHPE-lipid coated particles. As described in Example 2 the stability of incorporation of the lipid was probed by preparing particles using two different procedures: (1) direct incorporation of a lipid (DHPE lipid labeled with a fluorescent dye) during microparticle formation, and (2) physisorption of a lipid (DHPE labeled with a fluorescent dye) onto pre-formed microparticles. The lipid-containing microspheres produced from both methods were analyzed with fluorescence microscopy. Stability of incorporation was tested by treating the microparticles with repeated washings with water or a sodium dodecyl sulfate solution. Images from the microscopy are shown in FIGS. 2-3.

FIGS. 2A-2C show the brightfield images of poly(lactic acid) microparticles formed in the presence of the fluorescently-labeled lipid after washing with water (FIG. 2A), after washing once with sodium dodecyl sulfate (FIG. 2B), and after washing three times with sodium dodecyl sulfate (FIG. 2C). FIGS. 2D-2F are fluorescence images of the same microparticles after the same treatments; specifically, after washing with water (FIG. 2D), after washing once with sodium dodecyl sulfate (FIG. 2E), and after washing three times with sodium dodecyl sulfate (FIG. 2F). The brightfield images (FIGS. 2A-2C) show that the washings do not disrupt the structure of the microspheres. Fluorescence images (FIGS. 2D-2F) show that the fluorescently-labeled lipid remains, even after washing.

FIGS. 3A-3C show the brightfield images of poly(lactic acid) microparticles with fluorescently-labeled lipids physisorbed onto the preformed microparticles after washing with water (FIG. 3A), after washing once with sodium dodecyl sulfate (FIG. 3B), and after washing three times with sodium dodecyl sulfate (FIG. 3C). FIGS. 3D-3F are fluorescence images of the same microparticles after the same treatments; specifically after washing with water (FIG. 3D), after washing once with sodium dodecyl sulfate (FIG. 3E), and after washing three times with sodium dodecyl sulfate (FIG. 3F). The images show that the lipid, when attached to the microparticles by adsorption, was easily washed off by sodium dodecyl sulfate treatment (FIGS. 3D, 3F). The data presented in FIGS. 2-3 show that the lipid is stably incorporated into the particles when the lipid is present during formation of the particles.

The particles preferably additionally include a lipopolymer modified to include a ligand, forming a lipid-polymer-ligand conjugate, also referred to herein as a ‘lipopolymer-ligand conjugate’. The ligand can be a therapeutic molecule, such as a drug or a biological molecule having activity in vivo, a diagnostic molecule, such as a contrast agent or a biological molecule, or a targeting molecule having binding affinity for a binding partner, preferably a binding partner on the surface of a cell or extracellular matrix, or circulating in the blood stream. A preferred ligand has binding affinity for the surface of a cell and facilitates entry of the particle into the cytoplasm of a cell via internalization. The ligand in particles that include a lipopolymer-ligand is oriented outwardly from the particle surface, and therefore available for interaction with its binding partner or cognate receptor.

A variety of ligands can be attached to the lipopolymer, and methods for attaching ligands to lipopolymers are known, where the polymer can be functionalized for subsequent reaction with a selected ligand. (U.S. Pat. No. 6,180,134; Zalipsky, S. et al., FEBS Lett., 353:71 (1994); Zalipsky et al., Bioconjugate Chem., 4:296 (1993); Zalipsky et al., J. Control. Rel., 39:153 (1996); Zalipsky et al., Bioconjugate Chem., 8(2):111(1997); Zalipsky, S. et al., Meth. Enzymol., 387:50 (2004)). Functionalized polymer-lipid conjugates can also be obtained commercially, such as end-functionalized PEG-lipid conjugates (Avanti Polar Lipids, Inc.). The linkage between the ligand and the polymer can be a stable covalent linkage or a releasable linkage that is cleaved in response to a stimulus, such as a change in pH or presence of a reducing agent.

The ligand can be a molecule that has binding affinity for a cell receptor or for a pathogen circulating in the blood. The ligand can also be a therapeutic or diagnostic molecule, in particular molecules that when administered in free form have a short blood circulation lifetime. In one embodiment, the ligand is a biological ligand, and preferably is one having binding affinity for a cell receptor. Exemplary biological ligands are molecules having binding affinity to receptors for CD4, folate, insulin, LDL, vitamins, transferrin, asialoglycoprotein, selectins, such as E, L, and P selectins, Flk-1,2, FGF, EGF, integrins, in particular, α₄β₁ αᵥβ₃, αᵥβ₁ αᵥβ₅, αᵥβ₆ integrins, HER2, and others. Preferred ligands include proteins and peptides, including antibodies and antibody fragments, such as F(ab′)₂, F(ab)₂, Fab′, Fab, Fv (fragments consisting of the variable regions of the heavy and light chains), and scFv (recombinant single chain polypeptide molecules in which light and heavy variable regions are connected by a peptide linker), and the like. The ligand can also be a small molecule peptidomimetic. It will be appreciated that a cell surface receptor, or fragment thereof, can serve as the ligand. Other exemplary targeting ligands include, but are not limited to vitamin molecules (e.g., biotin, folate, cyanocobalamine), oligopeptides, oligosaccharides. Other exemplary ligands are presented in U.S. Pat. Nos. 6,214,388; 6,316,024; 6,056,973; 6,043,094, which are herein incorporated by reference. A recent review can be found in Zalipsky, S. et al., Meth. Enzymol., 387:50 (2004).

It will be appreciated that particles can be prepared to include both a lipopolymer and a ligand-lipopolymer; i.e., particles where only a portion of the hydrophilic chains in the lipid-polymer conjugate bear a ligand. It will also be appreciated that particles can be prepared to include two or more different ligands. It will also be appreciated that the particles can be prepared to include two or more different lipopolymers and/or ligand-lipopolymers where the polymer chains in the two or more different lipid-polymer conjugates are (i) different polymer or (ii) polymer of differing molecular weight. For example, particles comprising a first lipopolymer having a first molecular weight and a second lipopolymer having a second molecular weight can be prepared. Either or both of the first and second lipopolymers can include a ligand. In particular, particles having a ligand-lipopolymer and a lipopolymer, where the molecular weight of the polymer in the ligand-lipopolymer is greater than the molecular weight of the polymer in the lipopolymer, are contemplated. As can be appreciated, such particles present the ligand in polyvalent fashion for interaction with a target, unhindered by the shorter, lower molecular weight polymer chains in the lipopolymer. A specific example is a microparticle comprised of poly(dl-lactic acid) and of mPEG₂₀₀₀-DSPE and ligand-PEG₃₃₅₀-DPSE. Another specific example is a nanoparticle comprised of poly(1-lactic acid) and of mPEG₅₀₀₀-DSPE and ligand-PEG₂₀₀₀-DPSE. In this second example, the ligand is masked or shielded by the longer mPEG₅₀₀₀ polymer chains.

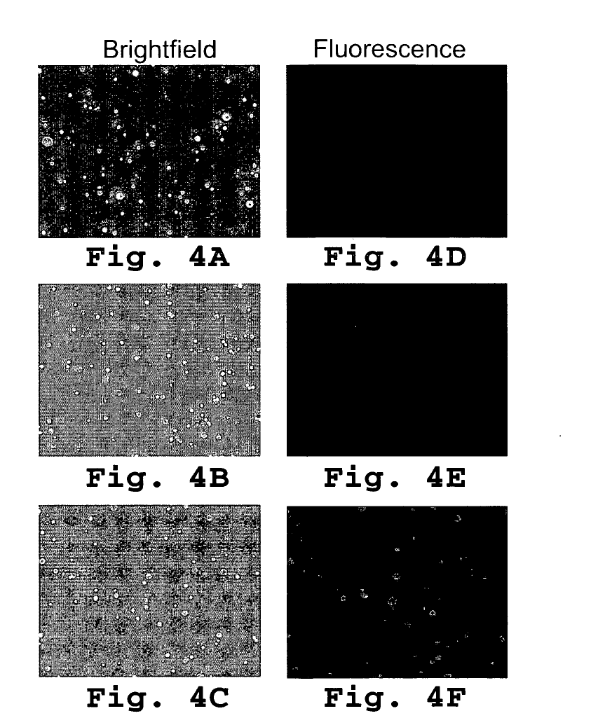

Particles comprising a lipid-polymer-ligand conjugate were prepared as described in Example 3. Microparticles of poly(lactic acid) and biotin-mPEG-DSPE were prepared by adding the ligand-lipopolymer to a solution of poly(lactic acid). The ligand-bearing microparticles were recovered and characterized using a receptor-ligand binding assay (Example 3D). Binding of the biotin-bearing microparticles with streptavidin was used to confirm that polymer-lipids functionalized with ligands are incorporated into the microspheres. The biotin-bearing microparticles were incubated with fluorescently-labeled streptavidin and observed under both brightfield and fluorescence microscopy. Comparative particles of poly(lactic acid) and of poly(lactic acid) (no lipopolymer) and mPEG-DSPE (no ligand), similarly incubated with streptavidin, were also observed. The images are shown in FIGS. 4A-4F.

FIGS. 4A-4C show the brightfield images for poly(lactic acid) microparticles (FIG. 4A), for poly(lactic acid) and mPEG-DSPE microparticles (FIG. 4B), and for poly(lactic acid) and biotin-PEG-DSPE microparticles (FIG. 4C) incubated with fluorescently-labeled streptavidin and with bovine serum albumin. FIGS. 4D-4F show the fluorescence images of the particles (FIG. 4D corresponds to poly(lactic acid) microparticles; FIG. 4E corresponds to poly(lactic acid) and mPEG-DSPE microparticles, and FIG. 4F corresponds to poly(lactic acid) and biotin-PEG-DSPE microparticles). In the presence of BSA, streptavidin bound only to the microspheres that had biotin-PEG2000-DSPE incorporated.

The results in FIGS. 4A-4F show that ligand-lipopolymers, including ligand-PEG-lipids, incorporate well into the particles. The ligand-lipopolymer was stably incorporated into the particles, as was illustrated in FIGS. 3A-3F.

Another study was conducted to evaluate binding of the particles under flow to a model lipid substrate. As described in Example 4, a bilayer lipid substrate of egg phosphatidylcholine and biotin-egg phosphatidylcholine was formed in the channels of a microfluidic device. Streptavidin was then bound to the biotinylated bilayer. Microparticles with DSPE-PEG-biotin or DSPE-PEG-thioctic acid (PEG molecular weight of 3300 Daltons) as model lipid-polymer-ligand conjugates were prepared. The particles also included a lipid-polymer conjugate, DSPE-PEG, where the PEG had a molecular weight of 2000 or 5000 Daltons. A small amount of a fluorescent label was incorporated into the particles. The four microparticle compositions are summarized in Table 1.

TABLE 1

| | | Lipopolymer- | |
|---|---|---|---|
| Formulation | Biodegradable | Ligand | Lipopolymer |
| Designation | Polymer | Composition | Composition |
| 1 - biotin- | poly-dl-lactide | biotin- | mPEG₂₀₀₀- |
| PEG₃₃₀₀-DSPE/ | | PEG₃₃₀₀-DSPE | DSPE |
| mPEG₂₀₀₀-DSPE | | | |
| 2 - biotin- | poly-dl-lactide | biotin- | mPEG₅₀₀₀- |
| PEG₃₃₀₀-DSPE/ | | PEG₃₃₀₀-DSPE | DSPE |
| mPEG₅₀₀₀-DSPE | | | |
| 3 - thioctic acid.- | poly-dl-lactide | thioctic acid- | mPEG₂₀₀₀- |
| PEG₃₃₀₀-DSPE/ | | PEG₃₃₀₀-DSPE | DSPE |
| mPEG₂₀₀₀-DSPE | | | |
| 4 - thioctic acid- | poly-dl-lactide | thioctic acid- | mPEG₅₀₀₀- |
| PEG₃₃₀₀-DSPE/ | | PEG₃₃₀₀-DSPE | DSPE |
| mPEG₅₀₀₀-DSPE | | | |

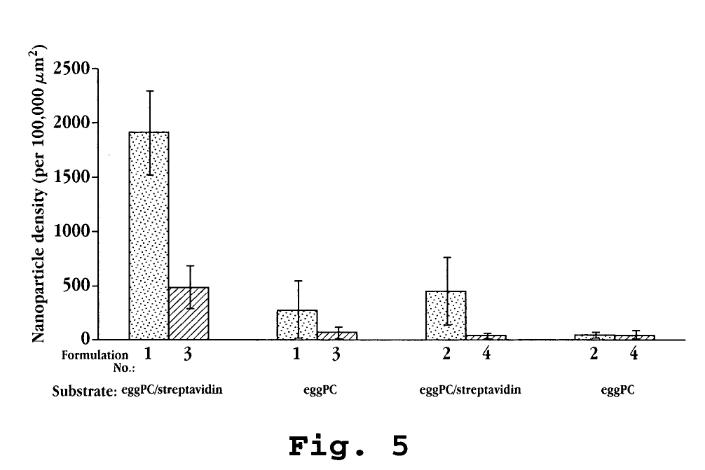

Each microparticle composition was introduced into a flow chamber for flow across a lipid bilayer substrate at a rate of 0.03 mL/minute (a shear rate of approximately 6 s⁻¹). A substrate of eggPC served as a control to the substrate containing streptavidin. The substrates were then imaged under an optical microscope for quantification of microparticle density. The results are shown in FIG. 5.

FIG. 5 is a bar graph of microparticle density per 100,000 micrometers for the four biotin-labeled microparticle compositions noted in Table 1, where the hatched bars correspond to the microparticles containing thioctic acid. Binding of the microparticles to a streptavidin-eggPC substrate and to an eggPC substrate (control) are shown. With respect to the microparticles containing biotin as the labeling ligand (dotted bars), the effect of relative length of the polymer on binding can be discerned from the data. The highest specific binding was observed with microparticle composition no. 1 comprised of microparticles containing, in addition to the biotin-PEG₃₃₀₀-DSPE, mPEG₂₀₀₀-DSPE. In microparticle formulation no. 1 the shorter (MW 2000 daltons) polymer, relative to the length of the polymer bearing the ligand (MW 3300 daltons), presents the ligand for binding with little interference from adjacent polymer chains. It is evident from microparticle formualtion no. 2 that the presence of polymer chains longer than the length of the chain bearing the ligand reduces the specific binding (˜4-fold reduction).

FIG. 5 also shows, in the hatched bars, the specific binding of formulation nos. 3 and 4, which contained thioctic acid as a target ligand. Similar to the biotin-containing microparticles, a reduced binding due to the presence of polymer chains having a higher molecular weight/longer length than that of the polymer bearing the ligand was observed. The shielding effect of mPEG₅₀₀₀-DSPE was significantly greater for the formulations containing thioctic acid compared to those containing biotin. It appears that in the presence of mPEG₅₀₀₀-DSPE the lower affinity thioctic acid was unable to achieve significant binding. The effect of ligand affinity on particle binding was also compared and found for biotin to be Kₐ˜10¹³ M⁻¹ and for thioctic acid to be Kₐ˜7×10⁷ M⁻¹.

### III. PARTICLE COMPOSITIONS AND METHODS OF USE

The particles described above serve as delivery vehicles or carrier platforms for a variety of agents incorporated into the particle core and/or carried on the distal end of the lipopolymer. The particles are typically formulated in a vehicle suitable for delivery. For example, the particles can be suspended in a pharmaceutical carrier, such as saline, for administration to a patient. The microparticles can be stored in dry or lyophilized form until administration, when, if desired, they are suspended in a fluid (liquid or gas) for administration.

The polymeric particles can be administered to humans and animals via a number of means including but not limited to orally, rectally, parenterally (intravenous, intramuscular, or subcutaneous), intravaginally, intraperitoneally, locally (in the form of powders, ointments, or drops), as a buccal delivery form, or nasal spray. In one embodiment, the particles are administered to a subject, with the proviso that the particles are not administered ocularly. The particles can also be incorporated into a medical device, such as a transdermal delivery device or a stent. It will be appreciated by those skilled in the art that the particles can be admixed with appropriate pharmaceutical diluents, carriers, excipients, or adjuvants suitably selected with respect to the intended route of administration and conventional pharmaceutical practices. For example, for parenteral injection, dosage unit forms may be utilized to accomplish intravenous, intramuscular or subcutaneous administration, and for such parenteral administration, suitable sterile aqueous or non-aqueous solutions or suspensions, optionally containing appropriate solutes to effectuate isotonicity, will be employed. Likewise for inhalation dosage unit forms, for administration through the mucous membranes of the nose and throat or bronchio-pulmonary tissues, suitable aerosol or spray inhalation compositions and devices will be utilized.

The size of the particles is selected according to the route of administration, the potency of the drug, the desired dosage, and other factors, such as the location of the intended target. Typically, nanoparticles generally have a diameter of about 1000 nm or less, preferably from about 5 nm to about 750 nm, and more preferably from about 10 nm to about 500 nm. Typically, microparticles will have a diameter of about 1000 μm or less, preferably from about 5 μm to about 750 nm, and more preferably from about 10 μm to about 500 μm.

The particles can be prepared to contain a variety of drugs and agents, as noted above. In particular, particles containing a peptide for treatment of a condition or of symptoms associated with a condition is contemplated. Exemplary preferred biologically active peptides for use include calcitonin, insulin, angiotensin, vasopressin, desmopressin, LH-RH (luteinizing hormone-releasing hormone), somatostatin, glucagon, somatomedin, oxytocin, gastrin, secretin, h-ANP (human atrial natriuretic polypeptide), ACTH (adrenocorticotropic hormone), MSH (melanocyte stimulating hormone), beta-endorphin, muramyl dipeptide, enkephalin, neurotensin, bombesin, VIP, CCK-8, PTH (parathyroid hormone), CGRP (calcitonin gene related peptide), endothelin, TRH (thyroid releasing hormone), growth hormones like erythropoietin, lymphokines like macrophage stimulating factor, and the like. The various polypeptides for use herein include not only the naturally occurring polypeptides themselves but also pharmacologically active derivatives and analogs thereof. Thus, for example, calcitonin includes not only naturally occurring products such as salmon calcitonin, human calcitonin, porcine calcitonin, eel calcitonin and chicken calcitonin, but also analogs. Similarly, LH-RH includes not only the naturally occurring product but also the pharmaceutically active derivatives and analogs thereof such as described in the literature (e.g., U.S. Pat. No. 3,917,825).

The particles can also be formulated to contain a small molecule drug or agent. Agents contemplated for use in the particles are widely varied, and non-limiting examples for therapeutic and diagnostic applications include steroids, immunosuppressants, antihistamines, non-steroidal anti-asthamtics, non-steroidal anti-inflammatory agents, cyclooxygenase-2 inhibitors, cytotoxic agents, gene therapy agents, radiotherapy agents, and imaging agents. In a preferred embodiment, the therapeutic agent is a cytotoxic drug, such as an anthracycline antibiotic, including but not limited to doxorubicin, daunorubicin, epirubicin, and idarubicin, including salts and analogs thereof. The cytotoxic agent can also be a platinum compound, such as cisplatin, carboplatin, ormaplatin, oxaliplatin, zeniplatin, enloplatin, lobaplatin, spiroplatin, ((−)-(R)-2-aminomethylpyrrolidine (1,1-cyclobutane dicarboxylato)platinum), (SP4-3(R)-1,1-cyclobutane-dicarboxylato(2-)-(2-methyl-1,4-butanediamine-N,N′)platinum), nedaplatin and (bis-acetato-ammine-dichloro-cyclohexylamine-platinum(IV)). The cytotoxic agent can also be a topoisomerase 1 inhibitor, including but not limited to topotecan, irinotecan, (7-(4-methylpiperazino-methylene)-10,11-ethylenedioxy-20(S)-camptothecin), 7-(2-(N-isopropylamino)ethyl)-(20S)-camptothecin, 9-aminocamptothecin and 9-nitrocamptothecin. The cytotoxic agent can also be a vinca alkaloid such as vincristine, vinblastine, vinleurosine, vinrodisine, vinorelbine, and vindesine. The entrapped therapeutic agent can also be an angiogenesis inhibitor, such as angiostatin, endostatin and TNFα.

### IV. EXAMPLES

The following examples further illustrate the subject matter described herein and are in no way intended to limit its scope. Various lipopolymer and ligand-lipopolymers are attainable following the published protocols, noted above, including Zalipsky, S. et al., Meth. Enzymol., 387:50 (2004).

### Example 1

### Preparation of Nanoparticles and Microparticles Including a Lipid

A. Microparticle Preparation

Poly (dl-lactide) (50 mg; Medisorb® 100DL High IV, Alkermes® (Cambridge, Mass.) MW 109 kD; Mn: 63 kD) was dissolved in ethyl acetate (2 mL) by sonication in a bath sonicator. After the poly(lactic acid) had dissolved, 40 μL of egg phosphatidylcholine (25 mg/mL in chloroform) and 10 μL of 1,2-dihexadecanoyl-sn-glycero-3-phosphoethanolamine (DHPE) labeled with Texas Red® (designated as Texas Red®-X DHPE where the X prefix refers to the fluorophore's extra julolidine rings; 1 mg/mL in chloroform; Molecular Probes, Eugene, Oreg.) were added. The solution was combined with 4 mL of 1% sodium cholate, and then homogenized for 15 seconds at 6000 rpm. The resulting suspension was then combined with 100 mL of 0.3% sodium cholate and stirred for 12-20 hours, evaporating the ethyl acetate through evaporation. The solution was then centrifuged and the pellets extracted, which were then washed three times with water at 4° C. FIGS. 1A-1B show brightfield and fluorescence images of the poly(lactic acid)-lipid DHPE microspheres (FIGS. 1A-1B, respectively). Microparticles of pure poly(lactic acid) were prepared according to the same procedure, and the brightfield and fluorescence images of these control particles are shown in FIGS. 1C-1D, respectively.

B. Nanoparticle Preparation

The procedure was carried out using the same methodology as in section A except the homogenization was carried out with a probe sonicator (Branson Ultrasonics Corp., Danbury, Conn.).

### Example 2

### Particle Stability

Stability of lipid incorporation into particles was tested by preparing microparticles by two procedures:

Procedure 1. Particles were prepared according to Example 1 by including DHPE labeled with the fluorescent dye Texas Red® in the poly(lactic acid) solvent solution prior to homogenization.

Procedure 2. Microparticles were also prepared from poly(lactic acid), i.e., excluding DHPE lipid, according to Example 1.

A lipid stock solutions consisting of egg PC supplemented with Texas Red®-X-DHPE was used to form lipid vesicles by the sonication method (Bayerl and Bloom, Biophys. J., 58: 357 (1990)). Briefly, the appropriate amount of each lipid was combined in 9:1 chloroform to methanol, dried with argon, and placed under vacuum for at least 2 hours. The lipid was hydrated with deionized water to a final concentration of 1 mg/mL and placed in a 50° C. oven for 20 minutes. The solution was then sonicated for 15 minutes with a Branson 450 tip sonicator (Branson Ultrasonics Corp., Danbury, Conn.) at 50% duty cycle and 3 output control in an ice bath to form vesicles.

The pre-formed poly(lactic acid) microspheres were mixed with the lipid vesicles containing fluorescently-labeled DHPE (Texas Red®-X-DHPE). The mixture was incubated for 3 minutes at 60° C., vortexed, and washed 3 times with water.

The lipid-modified microspheres prepared by Procedure 1 and Procedure 2 were subjected to three treatments (1) washing three times with water; (2) washing once with 10% sodium dodecyl sulfate (SDS); (3) washing three times with 10% SDS. The microparticles were visualized by Brightfield and Fluorescence microscopy. The images are shown in FIGS. 2-3. Scale bars are 100 μm.

### Example 3

### Microparticles with Biotin-PEG-DSPE

A. Preparation of Microparticles with Biotin-PEG₂₀₀₀-DSPE

Poly (dl-lactide) (50 mg; Medisorb® 100DL High IV, Alkermes® (Cambridge, Mass.) MW 109 kD; Mn: 63 kD) was dissolved in ethyl acetate (2 mL) by sonication in a bath sonicator. After the poly(lactic acid) had dissolved, 8.4 μL biotin-PEG₂₀₀₀-DSPE (10 mg/mL in chloroform) was added. The solution was combined with 4 mL of 1% sodium cholate, and then homogenized for 15 seconds at 6000 rpm. The resulting suspension was then combined with 100 mL of 0.3% sodium cholate and stirred for 12-20 hours, evaporating the ethyl acetate through evaporation. The solution was then centrifuged and the pellets extracted, which were then washed three times with water at 4° C.

B. Preparation of Microparticles with mPEG₂₀₀₀-DSPE

Microparticles comprised of poly(lactic acid) and of mPEG₂₀₀₀-DSPE were prepared according to the same procedure by substituting mPEG₂₀₀₀-DSPE for the biotin-PEG₂₀₀₀-DSPE.

C. Preparation of Poly(lactic acid) Microparticles Microparticles of pure poly(lactic acid) were prepared according to the procedure described in A. above, excluding addition of biotin-PEG₂₀₀₀-DSPE.

D. Receptor-Ligand Binding Assay

The microspheres formed according to the procedures in A-C above were brought up in phosphate buffered saline (PBS) with 2 mg/mL bovine serum albumin (BSA) and incubated for one hour. Specific binding of streptavidin was tested by adding 10 μL of streptavidin labeled with Texas Red® (Texas Red®-X-streptavidin) to each solution. Microspheres were then centrifuged (8000 rpm, 2 min), the supernatant was discarded, and the pellet was resuspended in the original solution (BSA-containing PBS). The microspheres were then observed with brightfield and fluorescence microscopy; the results are shown in FIGS. 4A-4F. Scale bars are 25 μm.

### Example 4

### Binding Studies Under Flow

A. Substrate Formation

Vesicle solutions of egg phosphatidylcholine (eggPC; Avanti Polar Lipids) and 5 mol % biotinylated-lipid (1,2-dipalmitoyl-sn-glycero-3-phosphoethanolamine-N-(Cap Biotinyl); Avanti Polar Lipids) in eggPC were formed through the sonication method. Briefly, lipids were combined in 9:1 chloroform to methanol, dried with argon, and placed under vacuum for at least 2 hours. Lipids were hydrated with de-ionized water to a final concentration of 1 mg/mL and placed in a 50° C. oven for 20 minutes. Solutions were then sonicated for 15 minutes with a probe sonicator at 50% duty cycle and 3 output control in an ice bath to form vesicles.

Parallel lanes of supported bilayers were formed using the vesicle fusion method (Brian et al., Proc. Natl. Acad. Sci. U.S.A., 81:6159 (1984)) in microfluidic channels. The microfluidic channels were formed using standard soft lithography techniques. Briefly, SU8-50 negative photoresist was spin coated onto silicon wafers to a thickness of roughly 100 μm. The coated wafers were then selectively exposed to UV light using a high resolution mask transparency and developed. Polydimethylsiloxane (PDMS) stamps were formed by curing Sylgard 184 at 70° C. for 5 hours on the silicon masters. Cured PDMS stamps were removed from the masters and inlets/outlets were punched with an 18 gauge blunt needle. Glass slides were plasma etched (PDC-32G, Harrick Scientific Corp., Ossining, N.Y.) for 2 minutes on high power under vacuum. PDMS stamps were then firmly pressed down against the glass slide forming a reversible, leak-tight seal. Vesicle solutions of eggPC and eggPC with biotinylated lipids were mixed 1:1 with PBS supplemented with 140 mM NaCl and vortexed. Each vesicle solution was then injected into an adjacent lane in the PDMS stamp and allowed to incubate at least 5 minutes at room temperature. Each channel was then flushed with de-ionized water to remove excess vesicles before removing the stamp in buffer (universally defined as PBS augmented with 0.5% (w/v) BSA).

After 10 minutes of incubation the buffer was exchanged, then 10 μL of streptavidin was added (1 mg/ml in PBS; Sigma). The streptavidin was allowed to incubate for at least 30 minutes before assembly into the flow chamber. Previous studies using FITC-streptavidin verified the homogeneous binding of streptavidin to the biotinylated bilayer, and that the streptavidin withstands shear rates rate in excess of 1000 s⁻¹.

B. Microparticle Preparation

Microparticles (MP) were prepared with the homogenization method as set forth above. The incorporated lipids consisted of 5 mol % biotin-PEG₃₃₀₀-DSPE or thioctic acid-PEG₃₃₀₀-DSPE in either mPEG₂₀₀₀-DSPE or mPEG₅₀₀₀-DSPE. The actual amounts of lipid incorporated are shown in the table below. In addition there was also 0.01 mg of Texas Red-DHPE incorporated into every batch for imaging. The mass of polylactic acid (PLA) was ˜20 mg for every batch.

| | | Lipopolymer- | |
|---|---|---|---|
| Formulation | Biodegradable | Ligand | Lipopolymer |
| Designation | Polymer | Composition | Composition |
| 1 - biotin- | poly-dl-lactide | biotin-PEG₃₃₀₀- | mPEG₂₀₀₀- |
| PEG₃₃₀₀-DSPE/ | (20 mg) | DSPE | DSPE |
| mPEG₂₀₀₀-DSPE | | 0.0789 mg/ | 0.97 mg/ |
| | | 0.0182 μmol | 0.345 μmol |
| 2 - biotin- | poly-dl-lactide | biotin-PEG₃₃₀₀- | mPEG₅₀₀₀- |
| PEG₃₃₀₀-DSPE/ | (20 mg) | DSPE | DSPE |
| mPEG₅₀₀₀-DSPE | | 0.0789 mg/ | 2.0 mg/ |
| | | 0.0182 μmol | 0.345 μmol |
| 3 - thioctic acid- | poly-dl-lactide | thioctic acid- | mPEG₂₀₀₀- |
| PEG₃₃₀₀-DSPE/ | (20 mg) | PEG₃₃₀₀-DSPE | DSPE |
| mPEG₂₀₀₀-DSPE | | 0.0783 mg/ | 0.97 mg/ |
| | | 0.0182 μmol | 0.345 μmol |
| 4 - thioctic acid- | poly-dl-lactide | thioctic acid- | mPEG₅₀₀₀- |
| PEG₃₃₀₀-DSPE/ | (20 mg) | PEG₃₃₀₀-DSPE | DSPE |
| mPEG₅₀₀₀-DSPE | | 0.0783 mg/ | 2.0 mg/ |
| | | 0.0182 μmol | 0.345 μmol |

After evaporation of solvent each microparticle solution was stored for use in flow experiments. For each experiment, 2 mL of a microparticle solution was centrifuged at 14,000 RPM for 15 minutes. Then the supernatant was removed, and the pellet was brought up in 2 mL deionized water. This water rinsing was repeated 3 times. Finally, the solution was centrifuged a fourth time and the pellet was re-suspended in 10 mL buffer. The microparticle suspension was then degassed and put into a sterile 10 mL plastic syringe. In addition, 20 mL of pure buffer was degassed and put into a sterile 20 mL syringe.

C. Flow Chamber Procedure

The buffer and a microparticle suspension were flushed through tubing and the flow chamber was assembled onto the prepared substrate under deionized water in a crystallization dish. Care was taken to avoid air bubbles in the chamber or upstream in the lines, and the substrate surface was always submerged to preserve supported bilayers. The flow chamber gasket provided laminar flow over an area 10 mm wide and 17 mm long. The thickness of the gasket was 0.010 inches (254 um), and the experimental height of the assembled flow chamber was 226±8 um (which did not vary during flow rates up to 6 mL/min). This experimental chamber height was used for calculation of shear rates according to the equation:

$$γ = \frac{6 Q}{{wh}^{2}} = \frac{6 Q * 1960}{{cm}^{3}}$$

where Q is the volumetric flow rate.

The following flow profiles of buffer and each microparticle suspension were then administered using an automated syringe pump (PHD 2000; Harvard Apparatus):
Step 1: Flow 2 mL buffer at 0.6 ml/min, shear rate ˜118 s⁻¹
Step 2: Flow 2 mL microparticle suspension at 0.03 ml/min, shear rate ˜6 s⁻¹
Step 3: Flow 10 mL buffer at 6 ml/min, shear rate ˜1176 s⁻¹

D. Image Analysis

The substrate was imaged under an optical microscope at 40×. Three fluorescence images were taken of different areas on each bilayer; the streptavidin-coated supported lipid bilayer and the control eggPC bilayer. Images were then analyzed with ImageJ software to determine the surface density of bound particles. Results are shown in FIG. 5.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A nanoparticle or microparticle comprised of a biodegradable polymer and of a first lipid-hydrophilic polymer-ligand conjugate, said conjugate incorporated into the nanoparticle or microparticle to provide an outer surface coating of hydrophilic polymer chains.

2. The nanoparticle or microparticle of claim 1 wherein said biodegradable polymer is selected from poly(lactic acid), poly(glycolic acid), poly(lactic-co-glycolic acid), and copolymers prepared from monomers of these polymers.

3. The nanoparticle or microparticle of claim 1, wherein said lipid-hydrophilic polymer-ligand conjugate is a lipid-poly(alkylene glycol)-ligand conjugate.

4. The nanoparticle or microparticle of claim 3, wherein the lipid-poly(alkylene glycol)-ligand conjugate is lipid-poly(ethylene glycol)-ligand.

5. The nanoparticle or microparticle of claim 4, wherein the lipid is 1,2-distearoyl-3-sn-glycerophosphoethanolamine (DSPE).

6. The nanoparticle or microparticle of claim 1, further comprising an agent.

7. The nanoparticle or microparticle of claim 6, wherein said agent is incorporated into the nanoparticle or microparticle.

8. The nanoparticle or microparticle of claim 6, wherein said agent is a therapeutic agent or a diagnostic agent.

9. The nanoparticle or microparticle of claim 6, wherein said agent is a peptide or protein.

10. The nanoparticle or microparticle of claim 1, further comprising a lipid-hydrophilic polymer conjugate.

11. The nanoparticle or microparticle of claim 10, wherein the hydrophilic polymer of the lipid-hydrophilic polymer conjugate has a molecular weight that is less than the molecular weight of the hydrophilic polymer of the lipid-hydrophilic polymer-ligand conjugate.

12. The nanoparticle or microparticle of claim 1, further comprising a second lipid-hydrophilic polymer-ligand conjugate having a ligand different from the ligand the first lipid-hydrophilic polymer-ligand conjugate.

13. The nanoparticle or microparticle of claim 12, wherein said ligand on said first or second conjugate is selected from the group consisting of biologically active ligands, targeting ligands, and diagnostic ligands.

14. A method for delivering an agent to a subject, comprising
administering to the subject a composition comprising nanoparticles or microparticles comprised of an agent, a biodegradable polymer, and a first lipid-hydrophilic polymer-ligand conjugate, said conjugate stably incorporated into the nanoparticles or microparticles to provide an outer surface coating of hydrophilic polymer chains and a ligand for interaction with a binding partner.

15. The method of claim 14, wherein said administering comprises administering nanoparticles or microparticles comprising a biodegradable polymer selected from poly(lactic acid), poly(glycolic acid), poly(lactic-co-glycolic acid), and copolymers prepared from monomers of these polymers.

16. The method of claim 14, wherein said administering comprises administering nanoparticles or microparticles comprising an agent having therapeutic activity.

17. The method of claim 16, wherein said administering comprises administering nanoparticles or microparticles comprising a protein or peptide.

18. The method of claim 14, wherein said administering comprises administering nanoparticles or microparticles comprising a conjugate comprised of a lipid-poly(alkylene glycol)-ligand.

19. The method of claim 18, wherein the lipid-poly(alkylene glycol) conjugate is lipid-poly(ethylene glycol)-ligand.

20. The method of claim 14, wherein said administering comprises administering nanoparticles or microparticles further comprising a second lipid-hydrophilic polymer-ligand conjugate having a ligand different from the ligand on the first lipid-hydrophilic polymer-ligand conjugate.

21. The method of claim 20, wherein said administering comprises administering nanoparticles or microparticles having a ligand selected from the group consisting of biologically active ligands, targeting ligands, and diagnostic ligands.

22. The method of claim 20, wherein said administering comprises administering nanoparticles or microparticles comprising at least two ligands, wherein said at least two different ligands are different from each other.

23. The method of claim 14, wherein said administering comprises administering nanoparticles or microparticles further comprising a lipid-hydrophilic polymer conjugate, wherein the hydrophilic polymer has a molecular weight that is less than the molecular weight of the hydrophilic polymer of the first lipid-hydrophilic polymer-ligand conjugate.
